(19) United States
(12) Patent Application Publication
(10) Pub. No.: US 2010/0234732 A1
(43) Pub. Date: Sep. 16, 2010

(54) METHOD FOR SIMULTANEOUSLY DETERMINING THE POSITION AND VELOCITY OF OBJECTS

(75) Inventor: Miroslaw Wrobel, Karlstadt (DE)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: Wittenstein AG, Ingersheim (DE)

(21) Appl. No.: 12/721,767

(22) Filed: Mar. 11, 2010

(30) Foreign Application Priority Data
Mar. 13, 2009 (DE) 10 2009 012 821.2

(51) Int. Cl. A61B 8/06; G01S 15/00; A61B 8/14

(52) U.S. Cl. 600/454; 367/90; 600/437; 600/481

(57) ABSTRACT

The present invention relates to a method for simultaneously determining the position and velocity of objects. Moreover the method comprising the steps of: 1) simultaneously generating a first signal for determining the velocity and a second signal for determining the position; 2) mixing the first signal with the second signal to form a mixed signal and subsequently transmitting the mixed signal towards an object using a transmitter, so that the mixed signal can be at least partially reflected by the object; and 4) evaluating an echo signal. Such a method may be used for various applications, such as monitoring blood vessel access and/or for pericardiocentesis, as well as for determining the blood flow velocity and the position of blood vessels.

The present invention relates to a method for simultaneously determining the position and velocity of objects. Moreover the method comprising the steps of: 1) simultaneously generating a first signal for determining the velocity and a second signal for determining the position; 2) mixing the first signal with the second signal to form a mixed signal and subsequently transmitting the mixed signal towards an object using a transmitter, so that the mixed signal can be at least partially reflected by the object; and 4) evaluating an echo signal. Such a method may be used for various applications, such as monitoring blood vessel access and/or for pericardiocentesis, as well as for determining the blood flow velocity and the position of blood vessels.

Methods for determining the velocity and the position of moving mediums and objects, respectively, are known in the state of the art. Hence, in the field of electromagnetic waves, for instance in the field of aviation, the position and the velocity of airplanes are determined using radar systems and in the field of medicine, the depth of objects and blood vessels as well as the flow velocity of flowing blood are determined using ultrasonic waves.

In fact, there are known approaches for simultaneously determining velocity and position, however, said approaches are subject to considerable restrictions with respect to resolution and measurability of the velocity. For, in order to enable enhanced resolution, for instance in the medical ultrasonic PW method, it is necessary to keep the transmitted wave packets at the smallest possible size (the resolution limit corresponds to half the size of the wave packet). However, this aspect in turn leads to an impaired detectability of the wave shift due to the Doppler effect, thus resulting in a deterioration of the determination of the velocity.

Moreover, for instance the ultrasonic CW method is known for determining the flow velocity in a relatively accurate fashion. However, this method does not allow for drawing conclusions as to the depth of the flow.

When transmitting pulses, for instance in the ultrasonic PW method or for instance in a combination of different methods, it is equally disadvantageous that it is thereby necessary to respectively wait until all of the echoes succeeding the transmitted pulse have subsided, in order to ensure an unambiguous determination of the depth and position of the echo origin.

As a consequence, it has hitherto been necessary to either wait until an idle time between the individual transmissions of various measuring pulses has elapsed, resulting in an increase of the total measurement time, or else to dispense with the accuracy in determining the depth and position and in determining the velocity of the flowing medium.

Moreover, in the simultaneous determination of the position and velocity using for instance the pulsed ultrasonic PW method, the velocity of the flowing medium conventionally cannot be determined in a continuous fashion, but can only be determined in a temporally separated fashion at intervals, resulting in that the velocity profile cannot be observed for instance during the idle times.

Said problems and drawbacks are encountered both in the field of ultrasound and in the field of electromagnetic waves.

Said restrictions have an adverse effect primarily on the region where blood vessels are accessed or on pericardiocentesis. Said access primarily serves for continuous intravascular hemodynamometry, for taking of blood samples destined for laboratory and blood gas analyses and for insertion of instruments (for instance in intercardiac catheter examinations).

The advancement of a guide wire of a catheter is frequently performed using radioscopy, in order to be able to observe the position of the wire. In this process, however, the patient is exposed to radiation. Subsequently, the puncture cannula is removed while the guide wire is fixed. Thereby, the blood vessel needs to be compressed at the puncture site, and it is necessary to pay strict heed to ensure that the position of the wire remains unchanged. As a function of the caliber of the catheter to be inserted (respectively of the drain or the port), it is necessary to widen the puncture channel beforehand using a dilator, in order to make insertion easier. Subsequently, the port or the catheter is advanced into its target position via the wire. In this process, it is advantageous to simultaneously monitor both the blood flow velocity and the position of the wire, in order to thusly for instance prevent the blood vessel from collapsing.

In the state of the art no method is known by means of which the simultaneous monitoring of the blood flow velocity and the position of the guide wire or else of the blood vessel via ultrasound is feasible.

As a consequence, it is an object of the present invention to propose a method for simultaneously determining the position and velocity of objects, which provides enhanced resolution, which involves reduced structural complexity and which enables simultaneous measurement both of the velocity and the position.

This object is attained by the present invention according to the teaching of the main claim.

Advantageous embodiments of the present invention are the subject-matter of the subclaims.

According to the invention, the existing drawbacks are overcome by the aspect that the method for simultaneously determining the position and velocity of objects comprises the following steps of:
simultaneously generating a first signal for determining the velocity and a second signal for determining the position,
mixing the first signal with the second signal to form a mixed signal and subsequently transmitting the mixed signal towards an object using a transmitter, so that the mixed signal can at least be partially reflected by the object,
evaluating an echo signal.

The generation of the signals can for instance be performed using signal generators or the like, wherein the signals are preferably generated in a separate fashion. Thereby, the first signal (hereinafter also referred to as velocity signal) for instance can be of a CW signal type and the second signal (hereinafter also referred to as positional signal) for instance can be of a signal type which is suitably modulated in terms of correlation.

Then, the velocity signal and the positional signal are initially mixed to form a mixed signal and the mixed signal is subsequently transmitted towards an object to be examined using a transmitter.

Thereby, subsequent to the generation of the signals and, where appropriate, to the processing of the signals, said signals for instance can be transferred to a mixing member which processes the velocity signal and the positional signal so as to form a mixed signal, and this single mixed signal is subsequently launched into the transmission medium via a transmitter. In this process, the mixing of the velocity signal and the positional signal is already performed prior to the transmission.

In contrast to an equally implementable alternative using two different transmitters for separately transmitting the velocity signal and the positional signal in the absence of prior mixing, the method involves reduced structural complexity, since in this example one transmitter can be omitted. Beyond that, accuracy can be enhanced as a result of the aspect that path differences due to transmitters placed at a distance or due to the spatial incoherency of transmitters can be prevented.

By transmitting a mixed signal which contains the information both of the first signal and of the second signal, a simultaneous determination both of the velocity and of the position is made possible, since both signals, i.e. the first signal and the second signal, reach the object simultaneously rather than in a temporally separated fashion.

By means of the simultaneous mixed transmission both of the velocity signal and of the positional signal as a mixed signal, two physical parameters, for instance velocity and position, can also be continuously determined at the same time. By means of this aspect it is possible to prevent dead times in the determination of physical parameters.

Subsequently, the mixed signal is emitted towards the object to be examined via a transmission medium (for instance via matter conducting sound waves, via air, or in case of electromagnetic waves, likewise via a vacuum).

According to a preferred embodiment, the method further comprises continuously receiving an echo signal reflected by the object.

According to another preferred embodiment, the evaluation comprises performing a velocity evaluation of the echo signal by taking into account the first signal and the Doppler effect, wherein velocity information as to the object is obtained as a result of said velocity evaluation.

Thereby, for instance in the velocity evaluation, the echo signal can be evaluated by taking into account the velocity signal and the Doppler effect. By taking into account the velocity signal in the evaluation of the echo signal and the Doppler effect, the informational content of the velocity signal can be isolated from the echo signal, so that accuracy and resolution can be influenced using mathematical operations, and velocity information as to the object can be obtained as a result of said velocity evaluation.

According to another preferred embodiment, the evaluation comprises performing a position evaluation of the echo signal by taking into account the second signal, wherein positional information as to the object is obtained as a result of said position evaluation.

When performing the position evaluation of the echo signal, for instance the positional signal can be taken into account, and hence, the informational content of the positional signal can be isolated from the echo signal, wherein accuracy and resolution can be influenced using mathematical operations. As a result of said position evaluation, positional information as to the object is obtained.

As a consequence, the echo signal is subjected to evaluations in order to obtain the physical parameters. Hence, both types of information, i.e. the velocity information and the positional information, can be obtained in a simultaneous and continuous fashion.

The field of application of the method is optional in terms of the signal range. According to a preferred embodiment, the signals can be generated in the form of ultrasonic waves, and, according to another preferred embodiment, the signals can be generated in the form of electromagnetic waves.

According to another preferred embodiment, the first signal is of a signal type which is suitable in terms of the Doppler effect, i.e. which is well suited for determining the frequency shift.

According to another preferred embodiment, the second signal is of a signal type which is suitably positioned in terms of pattern recognition, i.e. which is well suited for recognizing patterns during evaluations.

The form of the used signal is generally optional. According to another preferred embodiment, the second signal exhibits a constant frequency and is transmitted at intervals. Thereby, the positional signal can be easily generated and a pattern is impressed upon the signal by the transmission at intervals, by means of which pattern conclusions can be drawn as to the position of the object, for instance by observing echo transmission times.

The form in terms of the profile of the positional signal is basically optional. It has proved to be advantageous if the second signal obeys a function which can be mathematically correlated, in order to support the enhanced resolution option illustrated below. The better the correlation, the more accurately the resolution can be determined in the position determination.

According to another advantageous embodiment, the second signal exhibits a time-modulated frequency (FM). As a consequence, a pattern is imprinted upon the positional signal as well, by means of which it is equally possible to draw conclusions as to the echo transmission times and thusly as to the position of the object.

According to another advantageous embodiment, the mixed signal for instance obeys a mathematically smooth function. This aspect provides advantages in particular in the field of ultrasound, since by means of this aspect, response delays, for instance in piezoelectric ultrasound generators, can be prevented.

When selecting the velocity signal it has proved to be advantageous if the signal is continuously transmitted with a constant, invariable frequency, in order to be able to properly measure the Doppler shift with the aid of said frequency. The frequency of the velocity signal thereby should not overlap with the frequency/frequencies in the positional signal.

According to another preferred embodiment, the position evaluation comprises filtering of the echo signal with respect to the frequency of the second signal. By means of this aspect, the subsequent evaluation can be enhanced, for instance due to reduced noise.

According to another preferred embodiment, the positional evaluation comprises an analysis of the Doppler shift and/or the echo transmission time. Thereby, the echo signal, which, where appropriate, has been filtered beforehand, is subjected to an analysis. Said analysis can inter alia either encompass the observation of the Doppler shift or of the echo transmission time or else of both aspects.

According to another advantageous embodiment, the position evaluation comprises correlating the echo signal with the second signal and subsequently performing an analysis of the echo transmission time.

The quality of the correlation can have a decisive bearing on the resolution in terms of position determination, and the same can thusly be significantly enhanced. As an alternative to the correlation, convolution or else another method for pattern recognition can be utilized as well.

According to a preferred embodiment, the described method is used for monitoring blood vessel access and/or for pericardiocentesis.

According to another preferred embodiment, the described method is used for determining the blood flow velocity and the position of blood vessels.

Various embodiments of the present invention are illustrated in the drawings and will be specified by means of the following exemplary embodiments.

In the drawings:

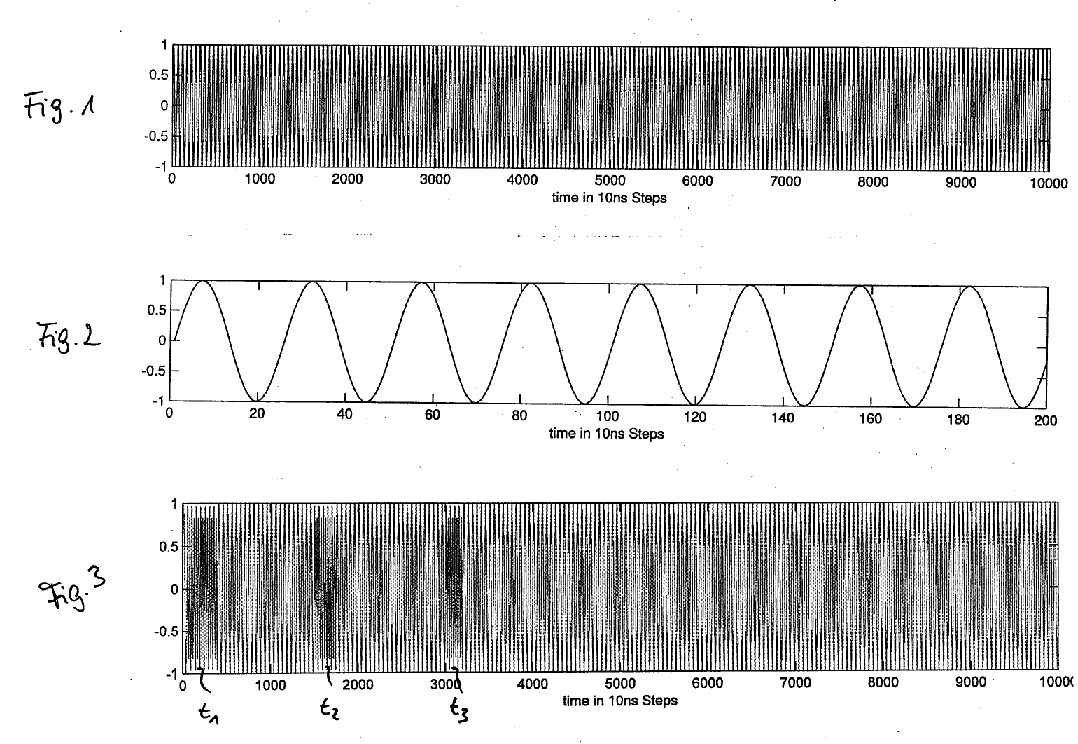

FIG. 1 illustrates a velocity signal with a constant frequency according to a first embodiment;

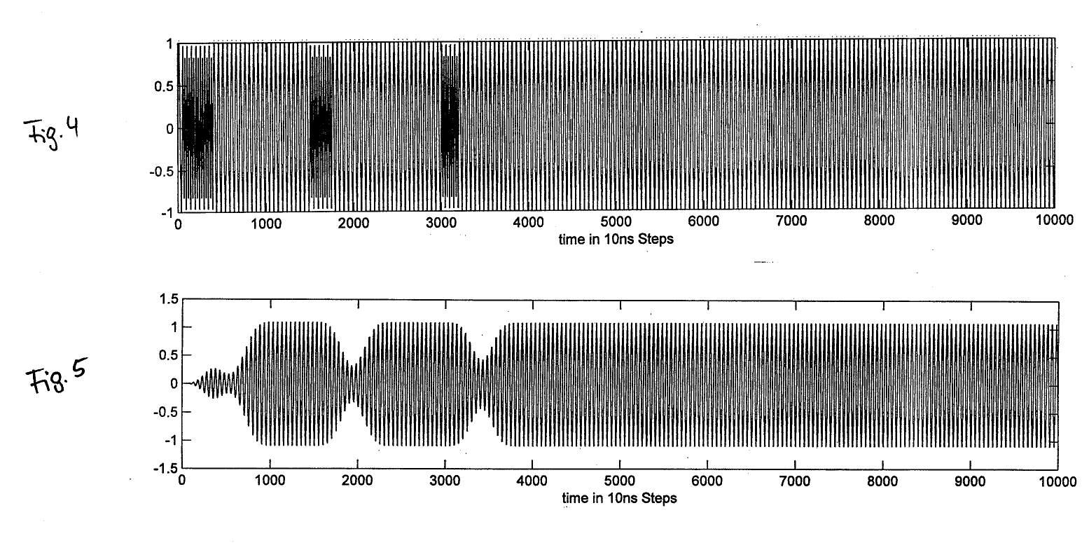

FIG. 2 illustrates a positional signal with a constant frequency transmitted at intervals according to a first embodiment;

FIG. 3 illustrates a mixed signal derived from the velocity signal according to FIG. 1 and the positional signal according to FIG. 2;

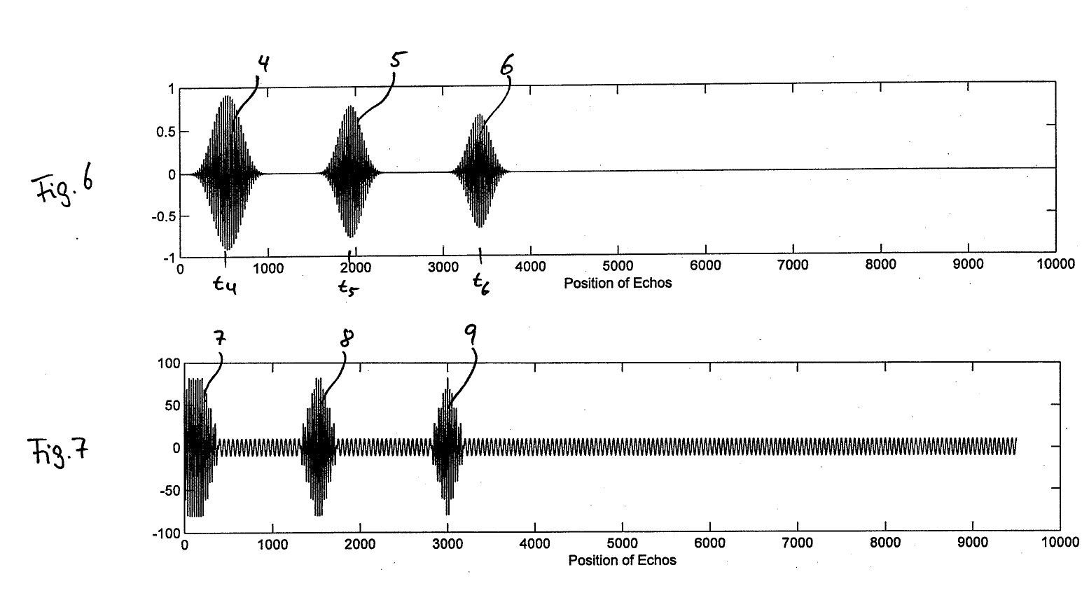

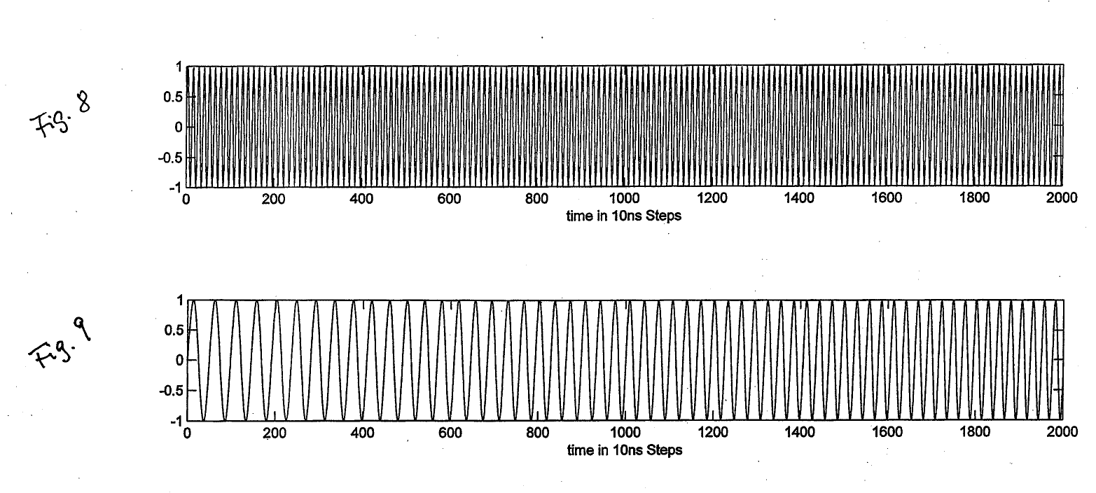

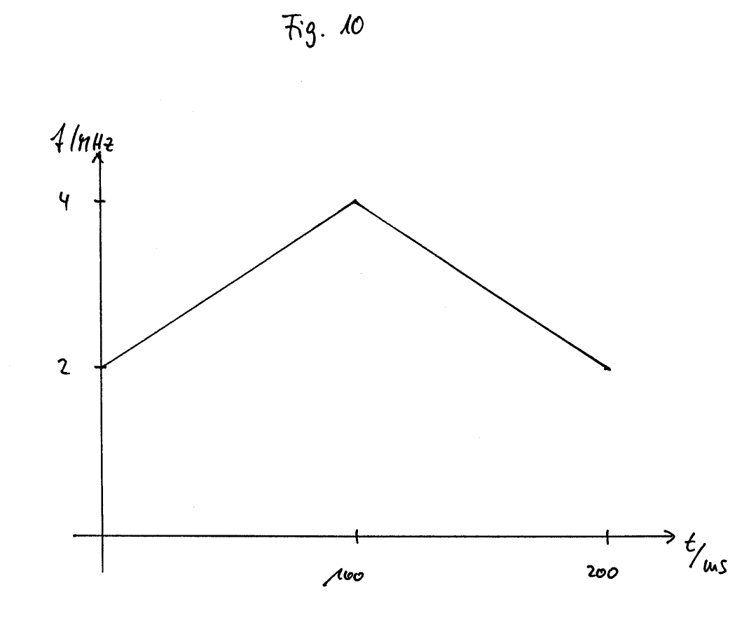

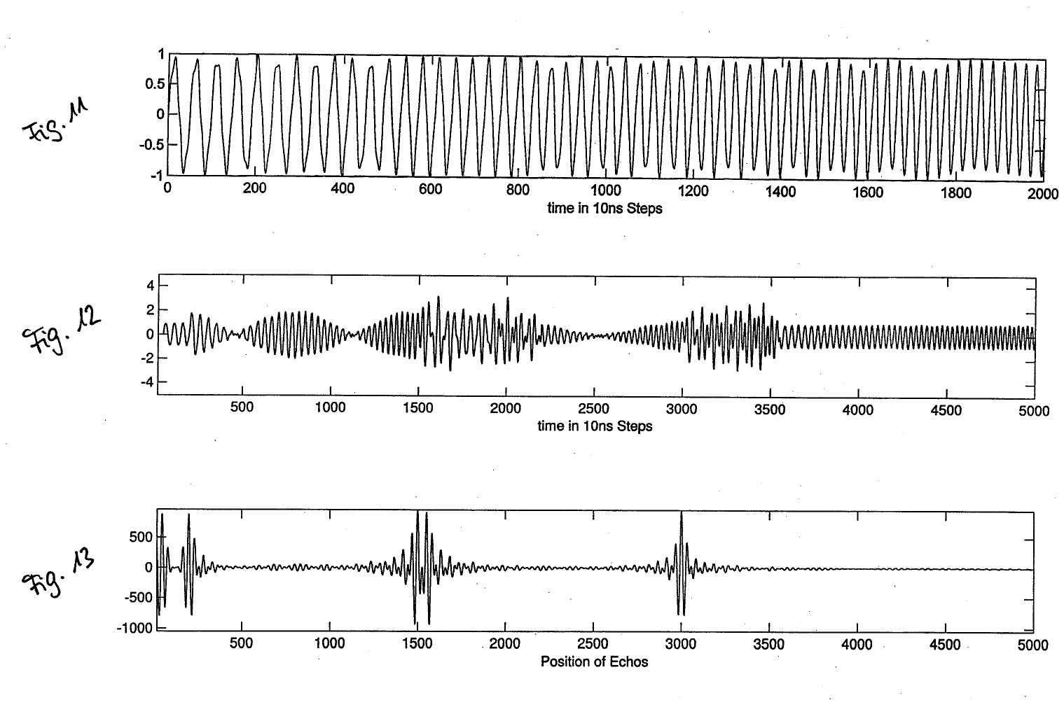

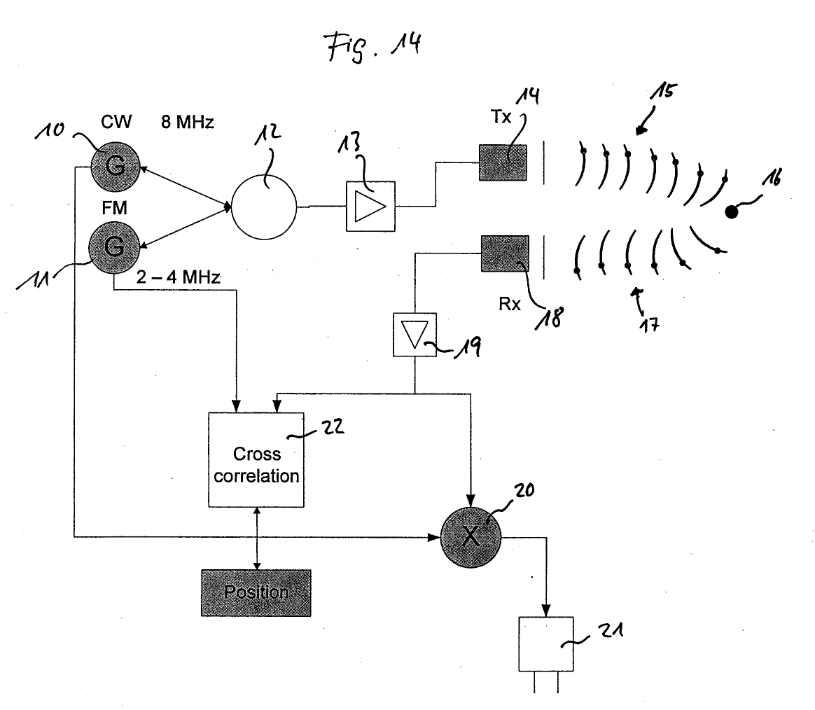

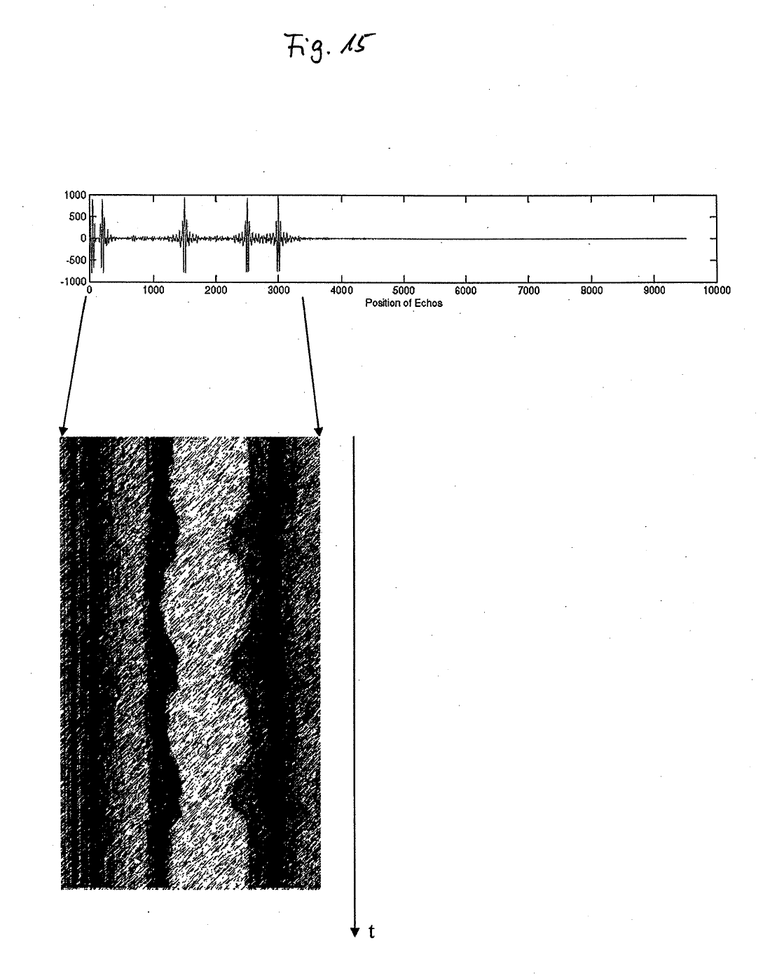

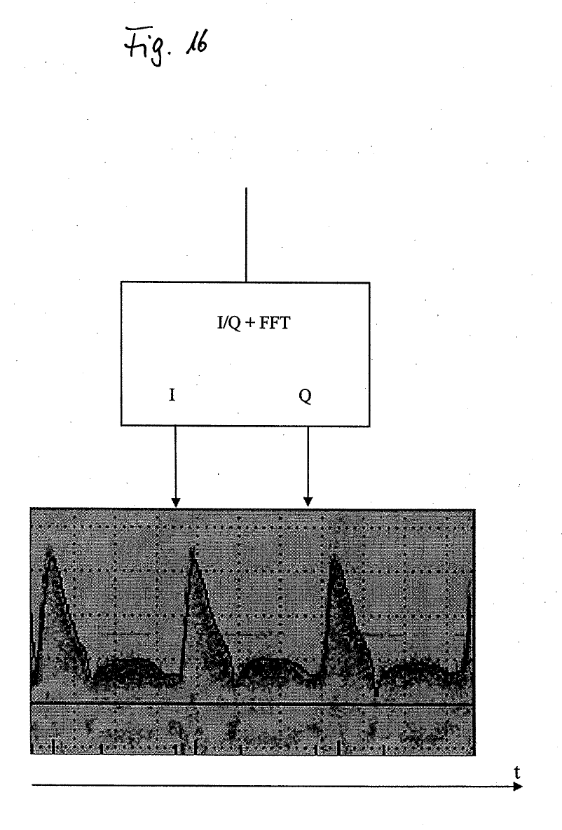

FIG. 4 illustrates an echo signal received subsequent to the transmission of the mixed signal according to FIG. 3;

FIG. 5 illustrates an echo signal according to FIG. 4 filtered with respect to the frequency of the velocity signal according to FIG. 1;

FIG. 6 illustrates an echo signal according to FIG. 4 filtered with respect to the frequency of the positional signal according to FIG. 2;

FIG. 7 illustrates an echo signal according to FIG. 4 correlated with the positional signal according to FIG. 2.

FIG. 8 illustrates a velocity signal with a constant frequency according to a second embodiment;

FIG. 9 illustrates a positional signal with a modulated frequency according to a second embodiment;

FIG. 10 illustrates an exemplary modulation function of the frequency modulation of the positional signal according to FIG. 9;

FIG. 11 illustrates a mixed signal derived from the velocity signal according to FIG. 8 and the positional signal according to FIG. 9;

FIG. 12 illustrates an echo signal received subsequent to the transmission of the mixed signal according to FIG. 11;

FIG. 13 illustrates an echo signal according to FIG. 12 correlated with the positional signal according to FIG. 9;

FIG. 14 illustrates the schematic structure of a device for performing the measuring method;

FIG. 15 illustrates a positional representation plotted over time by taking into account the correlated signal, similar to FIG. 13, referred to as M-mode;

FIG. 16 illustrates a representation of the blood flow velocity with the number of particles, referred to as Doppler.

FIG. 1 illustrates a velocity signal with a constant frequency, for instance of two Megahertz. This signal, in essence, corresponds to the input signal in a single ultrasound CW Doppler measurement. Said signal is continuously generated in a constantly successive fashion with a constant frequency.

FIG. 2 illustrates a positional signal likewise with a constant frequency, which is alternately generated with idle times. The frequency is thereby for instance set at four Megahertz. Similarly, as in the case of the ultrasonic PW measuring method, pulses are generated and transmitted using said signal, for instance pulses with a length of 2 μs. With the aid of the time required for detecting echoes on a pulse, conclusions can be drawn as to the reflection point, and hence as to the depth. It is problematic that short wave packets which in turn contain a small number of waves are required to ensure proper resolution. As a consequence, in the conventional ultrasonic PW method the accuracy of the velocity measurement using the Doppler effect is deteriorated.

FIG. 3 illustrates a mixed signal derived from the velocity signal according to FIG. 1 and the positional signal according to FIG. 2. Thereby, in the time intervals t₁, t₂ and t₃, respectively one pulse according to the positional signal according to FIG. 2 is mixed with the velocity signal according to FIG. 1. The mathematical operation of the mixing is basically optional.

At this stage, the method in principle corresponds to the simultaneous performance both of an ultrasonic CW measurement as well as of an ultrasonic PW measurement.

The mixed signal is transmitted towards the object to be examined and the medium to be examined, respectively, via a transmitter. At this site, the mixed signal is reflected and received as an echo signal.

The received echo signal is illustrated in FIG. 4.

FIG. 5 illustrates the echo signal according to FIG. 4 filtered to two Megahertz. Two Megahertz, in fact, precisely correspond to the frequency of the velocity signal according to FIG. 1, by means of which the velocity is supposed to be determined. Said filtered echo signal is subsequently compared with the transmitted velocity signal according to FIG. 1 and is subjected to an FFT analysis, in order to obtain the velocity to be determined using the frequency variations and the Doppler effect (see representation according to FIG. 16). Hence, the velocity is continuously determined without interruptions. This method is devoid of dead times at which the velocity remains unknown.

FIG. 6 illustrates the echo signal according to FIG. 4 filtered to the frequency of the positional signal according to FIG. 2, here to four Megahertz. Here, three echo packets 4, 5 and 6 are discernible. By means of the echo transmission times t₄, t₅ and t₆ conclusions can be drawn as to the position and depth of the reflected object and medium, respectively.

Moreover, the Doppler information contained in the echo packets 4, 5 and 6 allows for drawing conclusions as to the velocities at the reflection sites. In medical diagnostics, the signal could be represented as Tissue Doppler. This aspect does, however, entail the aforementioned drawbacks, i.e. that the resolution is considerably restricted and that the measurement is subject to various dead time intervals.

Irrespective of whether said Doppler evaluation is performed or is not performed due to said signal, the highly accurate velocity information from the signal according to FIG. 5, and besides, the positional information from the signal according to FIG. 6 is received in either case. Consequently, both parameters can be continuously obtained with a single measurement.

In addition, a moderate degree of accuracy of the position determination due to said signal is discernible, since the echo packets 4, 5 and 6 feature a to certain width. Said width is in particular dependent upon the length of the wave packets transmitted with the signal 2 according to FIG. 2.

FIG. 7 illustrates the echo signal according to FIG. 4 correlated with the positional signal according to FIG. 2.

The resemblance of said signal to the filtered signal according to FIG. 6 is discernible. Performing a Doppler evaluation at said correlated signal will no longer be possible though. However, due to the velocity determined with a high degree of accuracy, this aspect is relatively uncritical.

Conclusions as to the depth of the reflection site can be drawn from said signal in the same way as from the filtered signal according to FIG. 6. The echo packets 7, 8 and 9, however, are discernibly narrower than the echo packets 4, 5 and 6 from the filtered signal according to FIG. 6. Hence, the resolution in determining the depth and position of the reflection site and of the medium/object, respectively, has already been enhanced by means of the correlation in the evaluation.

FIG. 8 illustrates a velocity signal according to a second embodiment with a constant frequency of for instance eight Megahertz in the situation at hand. The signal is continuously generated and transmitted with said frequency in an uninterrupted fashion.

FIG. 9 illustrates a positional signal according to the second embodiment. The signal can be continuously generated and transmitted in an uninterrupted fashion, but can likewise be generated and transmitted at time intervals in an alternating fashion with an idle time.

In this example, the positional signal is frequency modulated with initially two Megahertz with a linear increase to four Megahertz and then again with a linear decrease to two Megahertz. Subsequent to the generation of such a frequency ramp, the signal is initially interrupted for the duration of an idle time and is thereafter generated and transmitted again for the duration of the frequency ramp.

FIG. 10 illustrates the frequency modulation function of the positional signal according to FIG. 9. Thereby, the linear increase of initially two Megahertz to four Megahertz, and at the end of the ramp, a decrease to two Megahertz is discernible. The selection of the modulation function is basically optional. Restrictions can be imposed for instance by mechanical requirements in terms of response delays in piezoelectric crystal generators, which aspect would, however, be unproblematic in generating electromagnetic waves.

It is particularly advantageous if the frequency modulation allows a distinct identification mark to be imprinted upon the positional signal. This aspect is reflected in a good resolution quality subsequent to the correlation.

FIG. 11 illustrates a mixed signal derived from the velocity signal according to FIG. 8 and the positional signal according to FIG. 9. Said mixed signal is subsequently conducted to a transmitter, in order to be likewise transmitted towards the medium/object to be examined, as in the case of the first embodiment.

FIG. 12 illustrates the echo signal reflected by the medium/object subsequent to the transmission of the mixed signal according to FIG. 11.

The evaluation using the Doppler shift can be performed at said signal in the same manner as performed at the signal according to FIG. 4, where appropriate, subsequent to the filtering to the frequency of the signal according to FIG. 8, in order to obtain the velocity of the medium/object.

FIG. 13 illustrates the echo signal according to FIG. 12 correlated with the positional signal according to FIG. 9.

The evaluation of the position/depth is performed at said signal via the transmission time of the echo packets in the same manner as performed at the signal according to FIG. 7 of the first embodiment.

The smaller size of the bandwidth of the reflected echo packet in contrast to the signal according to FIGS. 6 and 7 is clearly discernible. Hence, in this embodiment, the resolution and thusly the quality of the position determination is significantly enhanced. What is also clearly discernible in this example is the separation of actually 2 pulses in the range of 0 to approx. 250 and in the range of 1500, which are not yet separable in the signals according to FIGS. 6 and 7.

The resolution depends on the quality of the correlation, i.e. hence on the unambiguousness of the identification mark in the positional signal. The better the correlation of the positional signal, the higher the resolution in said signal.

FIG. 14 illustrates the structure of a device by means of which the measuring method according to the present invention can be performed. The velocity signal generator 10, for instance a CW signal generator, generates a signal with a constant frequency of eight Megahertz according to FIG. 8. The positional signal generator 11, for instance an FM signal generator, generates a positional signal with a frequency modulated from two to four Megahertz according to FIG. 9. Both signals are mixed in a mixing member 12 and are conducted to a transmitter 14 subsequent to amplification in the amplifier 13.

The transmitter 14 transmits the sound waves 15 towards the medium/object 16 to be examined, at which the sound waves 15 are subjected to reflection. The reflected sound waves 17 are subsequently received by a receiver 18 which, subsequent to amplification in the amplifier 19, conducts the received echo signal to the evaluation.

The velocity evaluation is performed in a mixer 20 by taking into account the velocity signal (generated by the signal generator 10), and the obtained velocity information is conducted to a representation member 21.

The positional evaluation is performed in a correlator 22 or the like by taking into account the positional signal generated by the positional signal generator 11. In the example at hand, the positional evaluation is inter alia performed by a cross-correlation with the positional signal. The positional information can be derived from the resultant signal, as described above.

FIG. 15 illustrates a representation option on the basis of a signal similar to that of FIG. 13. In the examination of, for instance pulsed blood vessels, said pulsation is rendered discernible by varying the transmission times of the echo packets. The transmission time of the echo packets and thusly of the positional information as to the reflection sites, for instance of the vessel walls, can be illuminated and plotted over time and can be represented in a so-called M-mode. Thereby, in the field of medical engineering, the treating person is constantly provided with information as to the vessel width and the depth/position thereof, respectively.

FIG. 16 illustrates another representation of the velocity information obtained as a result of the velocity evaluation. Thereby, the velocity information is for instance obtained as a real and imaginary part subsequent to performing a Fourier transformation, and is represented plotted over time.

In this example, the black line reflects the number of particles and the shadowing reflects the velocity of the particles.

On the basis of the present invention it is hence possible to simultaneously represent both the velocity information and the positional information without dead times. It is thusly possible to discern the number of particles and the velocity thereof and at which depth said flow is to be expected. It is even possible to allocate the flow to various blood vessels, for instance if several blood vessels are present in the field of view.

Special advantages are thusly provided in the field of ultrasound-based catheter examinations, since the present invention makes it possible to simultaneously represent the blood flow conditions prevailing in the blood vessels in the field of view as well as information as to the position of a guide wire. Thusly, the treating person is continuously provided with information as to the flow conditions surrounding the catheter tip and simultaneously as to the position of the catheter, and is consequently enabled to diagnose a collapse at an early stage, for instance via the flow curve pattern recognition and/or via the determination of the Reynolds number, and to thusly take counteractive measures.

1. A method for simultaneously determining the position and velocity of objects, said method comprising the steps of:
simultaneously generating a first signal for determining the velocity and a second signal for determining the position,
mixing the first signal with the second signal to form a mixed signal and subsequently transmitting the mixed signal towards an object using a transmitter, so that the mixed signal can be at least partially reflected by the object,
evaluating an echo signal.

2. The method according to claim 1,
wherein the method further comprises the step of continuously receiving an echo signal reflected by the object.

3. The method according to claim 1,
wherein the evaluation comprises the step of:
performing a velocity evaluation of the echo signal by taking into account the first signal and the Doppler effect,
wherein velocity information as to the object is obtained as a result of said velocity evaluation.

4. The method according to claim 1,
wherein the evaluation comprises the step of:
performing a position evaluation of the echo signal by taking into account the second signal, wherein positional information as to the object is obtained as a result of said position evaluation.

5. The method according to claim 1,
wherein the signals are generated in the form of ultrasonic waves.

6. The method according to claim 1,
wherein the signals are generated in the form of electromagnetic waves.

7. The method according to claim 1,
wherein the first signal is of a signal type which is suitable in terms of the Doppler effect.

8. The method according to claim 1,
wherein the second signal is of a signal type which is suitably positioned in terms of pattern recognition.

9. The method according to claim 1,
wherein the second signal exhibits a constant frequency and is transmitted at intervals.

10. The method according to claim 1,
wherein the second signal obeys a function which can be mathematically correlated.

11. The method according to claim 1,
wherein the second signal exhibits a time-modulated frequency (FM).

12. The method according to claim 1,
wherein the mixed signal obeys a mathematically smooth function.

13. The method according to claim 4,
wherein the position evaluation comprises filtering of the echo signal with respect to the frequency of the second signal.

14. The method according to claim 4,
wherein the position evaluation comprises an analysis of the Doppler shift and/or of the echo transmission time.

15. The method according to claim 4,
wherein the position evaluation comprises correlating the echo signal with the second signal and subsequently analyzing the echo transmission time.

16. A method for monitoring blood vessel access and/or for pericardiocentesis comprising the steps of:
simultaneously generating a first signal for determining the velocity and a second signal for determining the position,
mixing the first signal with the second signal to form a mixed signal and subsequently transmitting the mixed signal towards a blood vessel or pericardium using a transmitter, so that the mixed signal can be at least partially reflected by the object,
evaluating an echo signal.

17. The method for monitoring blood vessel access and/or for pericardiocentesis according to claim 16,
wherein the method further comprises the step of continuously receiving an echo signal reflected by the blood vessel or pericardium.

18. The method for monitoring blood vessel access and/or for pericardiocentesis according to claim 16,
wherein the evaluation comprises the step of:
performing a velocity evaluation of the echo signal by taking into account the first signal and the Doppler effect,
wherein velocity information as to the blood vessel or pericardium is obtained as a result of said velocity evaluation.

19. The method for monitoring blood vessel access and/or for pericardiocentesis according to claim 16,
wherein the evaluation comprises the step of:
performing a position evaluation of the echo signal by taking into account the second signal, wherein positional information as to the blood vessel or pericardium is obtained as a result of said position evaluation.

20. The method for monitoring blood vessel access and/or for pericardiocentesis according to claim 16,
wherein the signals are generated in the form of ultrasonic waves.

21. The method for monitoring blood vessel access and/or for pericardiocentesis according to claim 16,
wherein the signals are generated in the form of electromagnetic waves.

22. The method for monitoring blood vessel access and/or for pericardiocentesis according to claim 16,
wherein the first signal is of a signal type which is suitable in terms of the Doppler effect.

23. The method for monitoring blood vessel access and/or for pericardiocentesis according to claim 16,
wherein the second signal is of a signal type which is suitably positioned in terms of pattern recognition.

24. The method for monitoring blood vessel access and/or for pericardiocentesis according to claim 16,
wherein the second signal exhibits a constant frequency and is transmitted at intervals.

25. The method for monitoring blood vessel access and/or for pericardiocentesis according to claim 16,
wherein the second signal obeys a function which can be mathematically correlated.

26. The method for monitoring blood vessel access and/or for pericardiocentesis according to claim 16,
wherein the second signal exhibits a time-modulated frequency (FM).

27. The method for monitoring blood vessel access and/or for pericardiocentesis according to claim 16,
wherein the mixed signal obeys a mathematically smooth function.

28. The method for monitoring blood vessel access and/or for pericardiocentesis according to claim 19,
wherein the position evaluation comprises filtering of the echo signal with respect to the frequency of the second signal.

29. The method for monitoring blood vessel access and/or for pericardiocentesis according to claim 19,
wherein the position evaluation comprises an analysis of the Doppler shift and/or of the echo transmission time.

30. The method for monitoring blood vessel access and/or for pericardiocentesis according to claim 19,
wherein the position evaluation comprises correlating the echo signal with the second signal and subsequently analyzing the echo transmission time.

31. A method for determining the blood flow velocity and the position of blood vessels comprising the steps of:
simultaneously generating a first signal for determining the velocity and a second signal for determining the position,
mixing the first signal with the second signal to form a mixed signal and subsequently transmitting the mixed signal towards a blood vessel using a transmitter, so that the mixed signal can be at least partially reflected by the blood vessel,
evaluating an echo signal.

32. The method for determining the blood flow velocity and the position of blood vessels according to claim 31,
wherein the method further comprises the step of continuously receiving an echo signal reflected by the blood vessel.

33. The method for determining the blood flow velocity and the position of blood vessels according to claim 31,
wherein the evaluation comprises the step of:
performing a velocity evaluation of the echo signal by taking into account the first signal and the Doppler effect,
wherein velocity information as to the blood vessel is obtained as a result of said velocity evaluation.

34. The method for determining the blood flow velocity and the position of blood vessels according to claim 31,
wherein the evaluation comprises the step of:
performing a position evaluation of the echo signal by taking into account the second signal, wherein positional information as to the blood vessel is obtained as a result of said position evaluation.

35. The method for determining the blood flow velocity and the position of blood vessels according to claim 31,
wherein the signals are generated in the form of ultrasonic waves.

36. The method for determining the blood flow velocity and the position of blood vessels according to claim 31,
wherein the signals are generated in the form of electromagnetic waves.

37. The method for determining the blood flow velocity and the position of blood vessels according to claim 31,
wherein the first signal is of a signal type which is suitable in terms of the Doppler effect.

38. The method for determining the blood flow velocity and the position of blood vessels according to claim 31,
wherein the second signal is of a signal type which is suitably positioned in terms of pattern recognition.

39. The method for determining the blood flow velocity and the position of blood vessels according to claim 31,
wherein the second signal exhibits a constant frequency and is transmitted at intervals.

40. The method for determining the blood flow velocity and the position of blood vessels according to claim 31,
wherein the second signal obeys a function which can be mathematically correlated.

41. The method for determining the blood flow velocity and the position of blood vessels according to claim 31,
wherein the second signal exhibits a time-modulated frequency (FM).

42. The method for determining the blood flow velocity and the position of blood vessels according to claim 31,
wherein the mixed signal obeys a mathematically smooth function.

43. The method for determining the blood flow velocity and the position of blood vessels according to claim 34,
wherein the position evaluation comprises filtering of the echo signal with respect to the frequency of the second signal.

44. The method for determining the blood flow velocity and the position of blood vessels according to claim 34,
wherein the position evaluation comprises an analysis of the Doppler shift and/or of the echo transmission time.

45. The method for determining the blood flow velocity and the position of blood vessels according to claim 34,
wherein the position evaluation comprises correlating the echo signal with the second signal and subsequently analyzing the echo transmission time.
